(19) United States
(12) Patent Application Publication
(10) Pub. No.: US 2011/0105874 A1
(43) Pub. Date: May 5, 2011

(54) METHOD FOR MEASURING BODY PARAMETERS

(75) Inventors: Bastiaan Feddes, Eindhoven (NL); Alexander Padiy, Eindhoven (NL); Cristian Presura, Eindhoven (NL)

(73) Assignee: KONINKLIJKE PHILIPS ELECTRONICS N.V., EINDHOVEN (NL)

(21) Appl. No.: 12/673,985

(22) PCT Filed: Aug. 12, 2008
(86) PCT No.: PCT/IB08/53221

§ 371 Date: Feb. 18, 2010

(30) Foreign Application Priority Data
Aug. 20, 2007 (EP) 07301314.6

(51) Int. Cl. A61B 5/05

(52) U.S. Cl. 600/372

(57) ABSTRACT

A system comprises a capacitive sensing unit for capturing an electrophysiological signal from a body part. The capacitive sensing unit includes a first electrode plate that forms a capacitor with the body part. Motion of the electrode plate with respect to the body part may be detected by a motion sensitive unit mechanically coupled to the capacitive sensing unit. The motion sensitive unit detects motion by self-mixing interferometry. Then, a processing unit rejects the electrophysiological signal if a great displacement of the electrode plate is detected.

### FIELD OF THE INVENTION

The invention relates to a system and method for the measurement of body parameters. The invention is particularly relevant to the medical field and the measurement of electrophysiological signals using capacitive sensors.

### BACKGROUND ART

Although capacitive sensors are based on a mature technology, there is a great potential for their use in the medical area. For example, experiments have shown that capacitive sensors are well adapted to the detection of electrophysiological body signals, typically, heart (ECG), muscle (EMG) or brain (EEG) signals. In principle, capacitive sensors work as follows: an electrode plate is placed on a body part and the human tissue acts as the other capacitor plate capable of capturing the electrical signal generated by the muscles. A great advantage of capacitive sensors is that, contrary to other widely deployed techniques, there is no galvanic contact with the skin. By consequence there is no need for skin preparation and no need for a sticky patch with conductive gel typically needed to establish a good electrical contact between the skin and the sensor. On top of these advantages, capacitive sensors have shown great results and great sensitivity in the detection of electrical impulses caused by body elements such as the brain, the heart or nerves. Although the capacitor sensors technology is promising, it has not been yet adopted by the industry in part due to its high sensibility to motion. Motion of the sensor or the body is known to cause interferences in the captured signals and greatly influences the measurement results.

Several solutions have been proposed to reduce motion artifacts. For instance, U.S. Pat. No. 6,807,438 proposes to detect and reduce the motion induced artifacts by intentionally increasing the separation of the electrode plate and the skin. The effective capacitance varies with the distance between the plate and the skin. By allowing an offset between the electrode plate and the skin, the variation of capacitance with the distance becomes less sensitive to motion. However a disadvantage of the proposed solution is that the overall sensitivity of the sensor to the probed electrical signals also decreases.

Another solution is proposed in WO2006066566 where a method is described in which an electrical signal of known frequency is injected into the human body. By measuring the variation of this specific frequency on the capacitive sensor, a rough estimate can be made on the variation in distance. In this way variations in the distance between the plate and the skin can be detected and possibly even corrected for.

An additional problem caused by motion is that static charges are generated when the sensor is moving relative to the skin. The industry often refers to this problem as the triboelectric effect. These static charges may cause temporary malfunctioning of the capacitive sensors, e.g. due to clipping of the electronics.

The above solutions all offer to detect motion and attempt to correct for it however these solutions are either accompanied with a loss in sensitivity of the capacitive sensors or may pose health risks. These solutions often seek to modify the structure of the capacitive sensors or their environment so that the effect of motion can be reduced. The modifications of the capacitive sensors are nevertheless often a trade off to a loss in the preciseness of the final measurement.

### SUMMARY OF THE INVENTION

It is an object of the invention to provide a reliable body parameters system that is based on capacitive sensors.

It is another object of the invention to detect motion of the sensor with respect to the body without affecting the sensor's sensitivity.

It is yet another object of the invention to correct for motion artifacts without altering the structure of the capacitive sensors.

The invention therefore relates to a system first comprising a capacitive sensing unit comprising an electrode plate forming in combination with a body part a capacitor for capturing an electrophysiological signal from the body part. A motion sensitive unit is mechanically coupled to the capacitive sensing unit and detects by self-mixing interferometry a motion of the electrode plate with respect to the body part. The system also comprises a processing unit for altering the electrophysiological signal on the basis of the detected motion of the electrode plate.

A system of the invention is equipped with a motion sensitive unit that optically detects the motion of the capacitive sensing unit, and more precisely of the electrode plate, relative to the skin. Because both units are mechanically coupled, both units are subject to the same motion either laterally or perpendicularly to the body. The motion sensitive unit is thus capable of detecting displacement of the capacitive electrode. As explained above, motion disturbs the measured electrophysiological signal and the invention proposes to alter the signal when motion is detected. The signal may be modified using a correction algorithm or in some instances, the generated physiological signal may even be fully rejected because of strong artifacts that cannot be possibly corrected for. In an embodiment of the invention, the electrophysiological signal is fully rejected when motion sensing unit detects and measures a displacement greater than a maximal allowable threshold value.

A great advantage of a device of the invention is therefore its low sensibility to motion. Indeed, the inventors have realized that by adding an optical unit that uses self-mixing interferometry to conventional capacitive sensors, even small displacements can be detected. Various embodiments of the optical units further permit to determine the actual displacement value which may be subsequently used to correct the measurement signal values.

Another advantage of the invention is that no altering of the capacitive sensing unit is needed and hence a device of the invention the inherent high sensitivity of capacitive sensors. The invention proposes a post-processing of the electrophysiological signal instead of a modification of the set up of a sensing unit of the prior arts systems. Post processing will not affect measurements when no motion is detected and thus, again, preserves the inherent high sensitivity of capacitive sensors.

In an embodiment of the invention, the optical motion sensitive unit includes a light source for illuminating the body part and a cavity at the light source where interference is created. Light scattered by the body part interferes with the light already present in the cavity of the light source causing an interference signal representative of the motion of the electrode plate with respect to the body part. This interference signal may also represent power fluctuation of the light source and may be monitored by measuring the light intensity of the light produce by the light source with a photodiode.

The invention further relates to a method for measuring body parameters comprising:

detecting an electrophysiological signal from a capacitor formed from an electrode plate in combination with a body part;

optically detecting a motion of the electrode plate with respect to the body part by optical means using self-mixing interferometry; and

altering the electrophysiological signal on the basis of the detected motion of the electrode plate.

The foregoing has outlined rather broadly the features and technical advantages of the present invention so that those skilled in the art may better understand the detailed description that follows. One should appreciate that he may readily use the conception and the specific embodiments disclosed as a basis for modifying or designing other structures for carrying out the same purposes of the present invention.

### BRIEF DESCRIPTION OF THE DRAWING

For a more complete understanding of the present invention, and the advantages thereof, reference is now made to the following description taken in conjunction with the accompanying drawings, wherein like numbers designate like objects, and in which:

FIG. 1 is a device of the invention;

FIG. 2 is another view of device of FIG. 1;

FIG. 3 is a capacitive sensing unit of a device of the invention; and,

FIG. 4 is a motion sensitive unit of a device of the invention.

### DETAILED DESCRIPTION

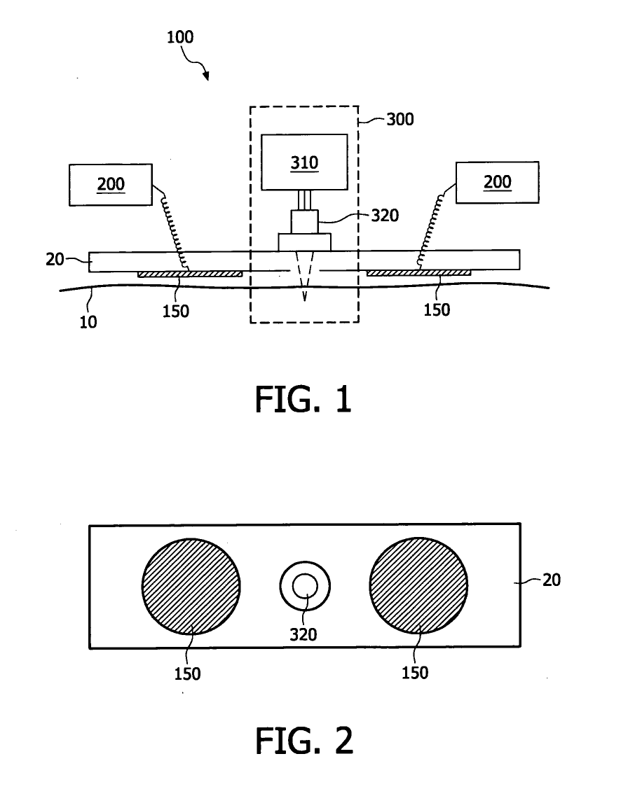

FIG. 1 shows a system 100 of the invention placed onto a body part 10 where medical examination is needed. Body part 10 is, for example, a patient's chest and in this example, system 100 captures the electrical activity of muscle fibers such as that of the heart in the case of an electrocardiogram. Device 100 may also be placed anywhere on the body, or else on the scalp to capture and record electrical impulses within the brain. Device 100 includes a conventional capacitive sensing unit that includes a pair of electrodes 150 and a processing unit 200. Device 100 further includes motion sensing unit 300 that is mechanically attached to electrodes 150 and processing unit 200. In FIG. 1, unit 300 is mechanically coupled with processing unit 200 and electrodes 150 via support 20. Alternatively, unit 300 is directly attached to electrodes 150 so that any displacement of the electrodes 150 relative to body part 10 causes a similar displacement of unit 300 with respect to the body part 10.

Motion sensing unit 300 optically detects movement of body 10. Examples of motion sensing unit 300 may be found in WO200237411 and WO200237124. Unit 300 includes a light source 320 built in with a cavity to permit the creation of interferences and processing unit 310. More details on unit 300 will be given in reference to FIG. 4.

FIG. 2 shows another view of device 100. FIG. 2 shows the side of support 200 that is placed directly onto the patient's skin. Support 200 may be made of a washable fabric or a flexible material where electrodes 150 are integrated. The optical motion sensing unit 200 is placed above an opening in support 200 so that light generated by light source 320 directly illuminates body part 10.

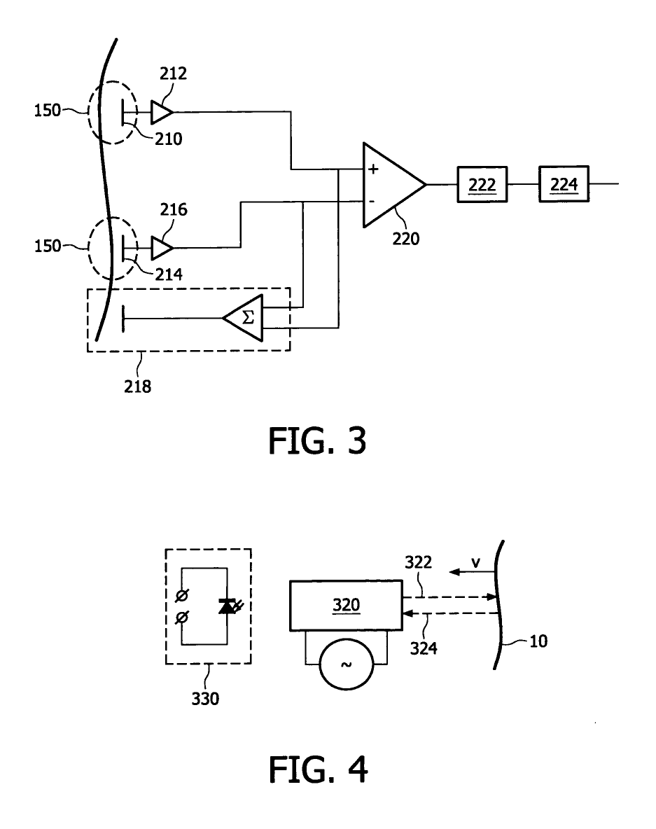

A more detailed description of the capacitive sensing unit formed by electrodes 150 and processing unit 200 now follows in reference to FIG. 3. A conventional sensing unit used in a device of the invention is of a bipolar set-up where two electrodes 150 are used in combination with a third reference electrode 218 to limit the common mode signals. Each electrode 150 is combined with an impedance converter 212, 216. The impedance converters 212 and 216 are preferably placed as close as possible to the individuals electrodes 150 so that minimal noise from the external environment is picked up by electrodes 150 due to their high impedance. The common mode signal is fed back to by the body via electrode 218 in order to limit the common mode signals on the signals generated by the electrodes 150. Unit 200 further comprises differential amplifier 220, an analog filter 222 and an analog to digital converter 224 for providing the electrophysiological signal representative of body signals such as the electric signals generated by body muscles. In another embodiment, capacitive sensing unit may also include an array of electrodes thereby permitting a greater sensitivity to the probed electrophysiological signal.

FIG. 4 is a motion sensitive unit 300 of the invention. The unit 300 works based on the principle of self-mixing interferometry. Basically light is emitted by a light source in laser cavity 320 and is then diffusively reflected by the body part 10 and the diffusely reflected light re-enters the laser cavity 320, sec arrows 322 and 324. The interference between the incoming light in the laser cavity and the light already present in laser cavity 320 creates power fluctuations of the laser. The power fluctuations may be measured with a photodiode 330 either placed outside the laser cavity or placed within the laser cavity. The interference pattern changes when body part 10 moves with respect to unit 300, or in this embodiment, when electrode 150 moves with respect to body part 10.

An application of unit 300 is the measurement of speed of displacement of the illuminated surface, body part 10, where the self-mixing interferometry is used for laser-Doppler velocimetry. When the body part 10 is moving with speed v the signal captured by the photodiode is modulated both in amplitude and frequency. The amplitude modulation is due to changes in the amount of light that is reflected into laser cavity 320, changing the interference pattern inside the laser cavity 320. Also the distance of the body part 10 to the laser influences this interference pattern. The frequency modulation is caused by the movement of body part 10. In an alternative embodiment, self mixing interferometry can also be performed also using two external cavities, instead of one. In this case an additional reflector, either a mirror or another portion of body part 10, is used as a reference reflector.

In operation, unit 300 is sensitive to motion of body part 10 by taking into account the Doppler shift that occurs when light is scattered by the moving body part 10. Assuming that the body part 10 moves with a constant velocity v in the direction of the laser and that the laser is not modulated, then the light scattered by the moving body part, e.g. an arm or the chest when the patient breathes or coughs, is Doppler shifted with the frequency Δf dependent on the velocity v according to the following equation:

$$Δ f = \frac{2 v}{λ} where λ is the wavelength of the laser .$$

However in practice, skin cannot always be modelled as a mirror as assumed in the above formulas. Thus the invention can be carried out in the IR wavelength, great results may also be obtained in the UV wavelength range where absorption by the body part 10 will be limited. Using low wavelengths, especially in the approximate range of 350-500 mn, show great results because the absorption increases quite dramatically then. As a result the optical probing depth will decrease compared to experiments obtained in the infrared. This means that the measurement of the displacements will become far more surface sensitive and absolute distance estimation gets more precise.

1. A system comprising:
a capacitive sensing unit comprising an electrode plate forming in combination with a body part a capacitor for capturing an electrophysiological signal from the body part;
a motion sensitive unit mechanically coupled to the capacitive sensing unit for detecting by self-mixing interferometry a motion of the electrode plate with respect to the body part; and,
a processing unit for altering the electrophysiological signal on the basis of the detected motion of the electrode plate.

2. The system of claim 1, wherein the processing unit rejects the electrophysiological signal when the detected motion lies within a predefined range.

3. The system of claim 1, wherein:
the motion sensitive unit comprises:
a light source for illuminating the body part;
a cavity at the light source where light scattered by the body part interferes with light transmitted by the light source causing an interference signal representative of the motion of the electrode plate with respect to the body part.

4. A method for measuring body parameters comprising:
detecting an electrophysiological signal from a capacitor formed from an electrode plate in combination with a body part;
optically detecting a motion of the electrode plate with respect to the body part by optical means using self-mixing interferometry; and
altering the electrophysiological signal on the basis of the detected motion of the electrode plate.
